(19) United States
(12) Patent Application Publication
(10) Pub. No.: US 2024/0016766 A1
(43) Pub. Date: Jan. 18, 2024

(54) PHARMACEUTICAL COMPOSITION FOR TREATMENT OF INNER EAR DISORDERS THROUGH LOCAL ADMINISTRATION IN THE TYMPANIC AREA

(71) Applicant: Alain MOUSSY, Paris (FR)

(72) Inventor: Alain MOUSSY, Paris (FR)

(21) Appl. No.: 18/034,960

(22) PCT Filed: Nov. 26, 2021
(86) PCT No.: PCT/EP2021/083195

§ 371 Date: May 2, 2023

(30) Foreign Application Priority Data
Nov. 26, 2020 (EP) 20306455.5

(51) Int. Cl. A61K 31/167; A61K 9/00; A61K 31/4535; A61K 31/352; A61K 31/428; A61K 31/198; A61K 31/485; A61K 31/517; A61K 31/137; A61K 31/00; A61K 31/573; A61K 31/4045; A61K 47/44; A61K 47/10

(52) U.S. Cl. CPC A61K 31/167; A61K 9/0046; A61K 31/4535; A61K 31/352; A61K 31/428; A61K 31/198; A61K 31/485; A61K 31/517; A61K 31/137; A61K 31/658; A61K 31/573; A61K 31/4045; A61K 47/44; A61K 47/10

(57) ABSTRACT

A pharmaceutical composition useful for the local administration onto the tympanic membrane and diffusion through the tympanic membrane without perforation of the membrane, for use in the treatment of an ear disorder or a neurological disorder such as hearing loss. Also, a device useful for the local administration onto the tympanic membrane of the pharmaceutical formulation of the invention.

### FIELD OF INVENTION

The present invention relates to a pharmaceutical composition for use in the treatment of an inner ear disorder or a neurological disorder. More specifically, this invention relates to the local administration of the pharmaceutical composition in the tympanic area, for use in the treatment of an inner ear or a neurological disorder: the invention provides a topical composition to be placed in the ear canal, capable of passing the active ingredient in the inner ear. The present invention also relates to a device useful for the local administration in the ear canal or onto the tympanic membrane of the pharmaceutical composition of the invention.

### BACKGROUND OF INVENTION

Treating inner ear disorders is not an easy task and is often carried out by systemic means. Local treatments are usually limited to injections. Direct injection into the inner ear of an auris formulation was described in the mid-1990s, for use in patients with Meniere's disease, autoimmune inner ear disease and sudden sensorineural deafness. However, the standard of care requires multiple injections over several days and causes great discomfort for patients. Also, such invasive route administration consisting of an injection through the tympanic membrane comes with some associated risks or side effects, such as residual perforation or increased susceptibility to infection.

More recently, a pharmaceutical composition for an administration into the inner ear via diffusion through the round windows membrane was described in the international patent publication WO 2010/048095 A2 (HOUSE EAR INST et al.) or in the US patent application US 2020/0230051 A1 (OTONOMY INC et al.). A sustained release formulation is administered on or near the round window via an intratympanic injection or through the tympanic cavity via a post-auricular incision. This disclosure shows explicitly that the patient still experiments an invasive gesture or a surgical intervention.

An object of the present patent application is to report the outstanding findings of the Applicant, now able to provide a non-invasive treatment of inner ear disorders and of neurological disorders, by providing a pharmaceutical active ingredient in the inner ear without any injection or invasive means. The purpose of the present invention is thus to provide a pharmaceutical composition, which, when applied topically in the tympanic area, is able to carry on the pharmaceutical active ingredient into the inner ear.

### SUMMARY

This invention thus relates to a pharmaceutical composition for administration into the ear canal or onto the tympanic membrane, for use in the treatment of inner ear disorders, comprising: at least one active agent and a pharmaceutically acceptable vehicle comprising: at least one diffusing agent selected from the group consisting of vegetable oils, oil/water serum bases, glycerine, alcohols, and a mixture thereof, and at least one solubilizing agent selected from the group consisting of terpenes and derivatives thereof.

According to one embodiment, the at least one active agent is selected from the group consisting of repair agents, ionic channels antagonists, glutamate receptors antagonists, dopamine agonists or precursors, psychotropic drugs, antioxidant compounds, myorelaxant agents, vasodilators, diuretic compounds, other agents blocking signal transmission, nutritional supplements, anti-edema compounds, and a mixture thereof. According to one embodiment, the at least one active agent is an ionic channel antagonist, wherein the ionic channel antagonist is a sodium channel antagonist, preferably lidocaine. According to one embodiment, the at least one active agent is a psychotropic drug, preferably selected from the group consisting of amitriptyline and cannabidiol. According to one embodiment, the at least one active agent is a glutamate receptor antagonist, preferably selected from the group consisting of dextromethorphan, gacyclidine and selurampanel. According to one embodiment, the at least one active agent is a dopamine agonist or precursor, preferably selected from the group consisting of levodopa (L-DOPA), pramipexole and ropinirole. According to one embodiment, the at least one active agent is a repair agent, wherein the repair agent is a Tropomyosin Receptor Kinase (TRK) agonist (such as a Tropomyosin receptor kinase B (TrkB) agonist or a Tropomyosin receptor kinase C (TrkC) agonist), preferably 7,8-dihydroxyflavone (7,8-DHF), or a growth factor, preferably neurotrophin-3 (NT3). According to one embodiment, the at least one active agent is an anti-edema compound, preferably dexamethasone. According to one embodiment, the at least one active agent is an antioxidant compound. In one embodiment, the at least one active agent is an antioxidant compound or a repair agent, preferably neurotrophin-3 (NT3), the at least one diffusing agent is α-(-)-bisabolol, and the at least one solubilizing agent is apricot oil. According to one embodiment, the at least one active agent is at a concentration ranging from 0.001% to 20% by weight based on the total weight of the composition, preferably from 0.01% to 10% by weight based on the total weight of the composition.

According to one embodiment, the vegetable oils are selected from the group consisting of almond oil, apricot oil, arachis oil, camellia oil, castor oil, coconut oil, cottonseed oil, evening primrose oil, grapeseed oil, jojoba oil, maize oil, olive oil, rapeseed oil, rosehip oil, safflower oil, sesame oil, soybean oil, sunflower oil, medium chain triglycerides (MCT), wheat germ oil, and a mixture thereof; preferably apricot oil. According to one embodiment, the oil/water serum base is Seraqua™. According to one embodiment, the alcohols are selected from the group consisting of propanediol, isopropanol, benzyl alcohol, ethanol, and a mixture thereof. According to one embodiment, the terpenes and derivatives thereof are sesquiterpene alcohols; preferably selected from the group consisting of α-(-)-bisabolol, linalool, and a mixture thereof; more preferably α-(-)-bisabolol.

According to one embodiment, the pharmaceutical composition is in the form of a liquid or gel formulation, preferably in the form of ear drops. According to one embodiment, the pharmaceutical composition is for administration in the tympanic area. According to one embodiment, the use comprises a step of administering the pharmaceutical composition into the ear canal or onto the tympanic membrane, preferably in the tympanic area. According to one embodiment, the pharmaceutical composition is administered at a volume ranging from 5 μl to 100 μl per ear.

According to one embodiment, the inner ear disorder is selected from otosclerosis; non-obliterative and obliterative otosclerosis involving oval window; cochlear otosclerosis; acoustic neuroma; bilateral and unilateral sensorineural hearing loss such as noise-induced hearing loss or aged-induced hearing loss such as presbycusis; bilateral and unilateral conductive hearing loss; bilateral and unilateral mixed conductive and sensorineural hearing loss; sudden idiopathic hearing loss; drug-induced hearing loss; single sided deafness; ear infection; ototoxicity; drug-induced ototoxicity; herpes zoster oticus; labyrinthitis; purulent labyrinthitis; labyrinthine fistula; Labyrinthine dysfunction; vestibular neuronitis; excitotoxicity; autoimmune inner ear disease; acute unilateral vestibulopathy; vestibular neuronitis; benign paroxysmal positional vertigo; vertigo of central origin; Meniere's disease; Meniere's syndrome and tinnitus. According to one embodiment, the inner ear disorder is selected from the group consisting of hearing loss and tinnitus. In one embodiment, the inner ear disorder is tinnitus.

In one embodiment, the at least one active agent is selected from the group consisting of 7,8-dihydroxyflavone (7,8-DHF) or levodopa (L-DOPA); and the inner ear disorder is tinnitus. In one embodiment, the at least one active agent is a growth factor or a Tropomyosin Receptor Kinase (TRK) agonist, such as a Tropomyosin receptor kinase B (TrkB) agonist or a Tropomyosin receptor kinase C (TrkC) agonist; and said inner ear disorder is selected from the group consisting of hearing loss and tinnitus.

The invention also relates to a device for the local administration of the pharmaceutical composition for use according to the invention, onto the tympanic membrane via the ear canal, the device comprising a pump, a reservoir containing the pharmaceutical composition for use according to the invention, and a catheter channel.

This invention also relates to a method for administering a therapeutically effective amount of an active agent to a patient in need thereof, for the treatment of an inner ear disorder, comprising administering ear drops into the ear canal or onto the tympanic membrane of the patient, the ear drops comprising the pharmaceutical composition for use according to the invention.

### DEFINITIONS

The definitions and explanations below are for the terms as used throughout the entire application, including both the specification and the claims.

In the present invention, the following terms have the following meanings:

The term “about” is used herein to mean approximately, roughly, around, or in the region of. The term “about” preceding a figure means plus or less 10% of the value of the figure. When the term “about” is used in conjunction with a numerical range, it modifies that range by extending the boundaries above and below the numerical values set forth by 10%.

The term “administration”, or a variant thereof (e.g., “administering”), means providing the active agent, alone or as part of a pharmaceutically acceptable composition, to the patient in whom/which the condition, symptom, or disease is to be treated or prevented.

The term “agonist” refers to a natural or synthetic compound which binds to the protein and stimulates the biological activation of the protein, and thereby the action of the protein.

The term “antagonist” as used herein means a compound that competitively or non-competitively binds to a receptor at the same site as an agonist (for example, the endogenous ligand) and has reversible and competitive binding affinity to a receptor without direct modulation of receptor signalling, but that nonetheless occupies the binding site of an agonist (for example, the endogenous ligand) to thereby block agonist-mediated receptor signalling.

The term “diffusing agent” refers to a chemical entity that facilitates the transport of at least one active agent across the tympanic membrane. In the meaning of this invention, the diffusing agent preferably proceeds through non-invasive transport of the active agent, especially the diffusing agent does not incur any perforation of the tympanic membrane.

The term “in the ear canal” is a shortcut for “into the ear canal and/or onto the tympanic membrane”. As the present invention is related to non-invasive treatment, in this context administration “into the ear canal” refers to topical administration into the outer part of the ear canal, i.e., the ear canal extending from the pinna to the outer tympanic membrane. Similarly, in the invention, administration “onto the tympanic membrane” refers to topical administration to the outer side of the tympanic membrane, i.e., the side of the tympanic membrane that face the outer ear canal.

The term “inner ear” refers to the part of the ear containing the cochlea, a bony, fluid-filled organ responsible for hearing, as well as the auditory nerve, which transmits signals from the cochlea to the brain. Also located in the inner ear are the vestibular canals that control a person's sense of balance.

The term “neurological disorder” includes diseases of the central and peripheral nervous system, i.e. the brain, spinal cord, cranial nerves, peripheral nerves, nerve roots, autonomic nervous system, neuromuscular junction, and muscles. It may refer to any disorder or disease affecting neurons, neurotransmitters, or neurotransmitter transporters. In the meaning of this invention, neurological disorders include epilepsy, Alzheimer's disease and other dementias, cerebrovascular diseases including stroke, migraine and other headache disorders, multiple sclerosis, Parkinson's disease, neuroinfections, brain tumours, traumatic disorders of the nervous system due to head trauma, and neuropsychiatric disorders such as schizophrenia and paranoia.

The term “non-invasive” refers to an administration, use or method not involving the introduction of solid objects such as medical instruments (needle, scalpel, etc.) into the body of a patient, especially through an organ or tissue of the patient. In particular, “non-invasive” means that the tympanic membrane of the patient is not perforated or otherwise penetrated. For example, administration “in the ear canal” as defined herein is non-invasive.

The term “oil of medium or light viscosity” refers to an oil displaying a viscosity lower than 40 centipoises measured at 20° C. in operating conditions where glycerine displays a viscosity of 800 centipoises. For example, the ratio of the viscosity of the oil to the viscosity of glycerine may be lower than 0.05 or lower than 0.01 or lower than 0.005 or lower than 0.001, both viscosities being measured in the same temperature and operating conditions.

The term “patient” refers to a warm-blooded animal, more preferably a human, who/which is awaiting the receipt of, or is receiving medical care or is/will be the object of a medical procedure.

The term “pharmaceutical composition” refers to a composition comprising an active agent in association with a pharmaceutically acceptable vehicle. A pharmaceutical composition is for therapeutic use, and relates to health. Especially, a pharmaceutical composition may be indicated for treating or preventing a disease.

The term “pharmaceutically acceptable vehicle” as used herein means a medium comprising at least the diffusing agent(s) and the solubilizing agent(s) and used as solvent or diluent in which the pharmaceutically active agent is formulated and/or administered. Non-limiting examples of pharmaceutical vehicles include creams, gels, lotions, solutions and liposomes.

The term “pharmaceutically acceptable” refers to compounds, materials, compositions and/or dosage forms which are, within the scope of sound pharmaceutical/medical judgment, suitable for use in contact with the tissues of human beings and animals without excessive toxicity, irritation, allergic response, or other problem or complication, commensurate with a reasonable benefit/risk ratio. Thus, the term “pharmaceutically acceptable vehicle”, as used herein, may comprise such suitable compounds and materials defined below that may be added to the composition to assist in satisfactory processing of the formulation or provide desirable physical characteristics to the formulation. For example, “pharmaceutically acceptable vehicle” may include, but is not limited to, any and all substances such as for example solvents, cosolvents, antioxidants, surfactants, stabilizing agents, emulsifying agents, buffering agents, pH modifying agents, preserving agents, antibacterial agents, antifungal agents, isotonifiers, dispersing agents, suspending agents, oils, lubricants and mixtures thereof. For human administration, preparations should meet sterility, pyrogenicity, general safety and purity standards as required by regulatory offices, such as, e.g., FDA Office or EMA.

The term “solubilizing agent” refers to a compound or mixture of compounds present in the pharmaceutically acceptable vehicle and wherein at least part of the active agent and/or the diffusing agent may be dissolved or suspended.

The expression “therapeutically effective amount” means level or amount of agent that is aimed at, without causing significant negative or adverse side effects to the target, (1) delaying or preventing the onset of an ear disease, disorder, or condition; (2) slowing down or stopping the progression, aggravation, or deterioration of one or more symptoms of the ear disease, disorder, or condition; (3) bringing about ameliorations of the symptoms of the ear disease, disorder, or conditions; (4) reducing the severity or incidence of the ear disease, disorder, or condition; or (5) curing the ear disease, disorder, or condition. A therapeutically effective amount may be administered prior to the onset of the ear disease, disorder, or condition, for a prophylactic or preventive action. Alternatively, or additionally, the therapeutically effective amount may be administered after initiation of the ear disease, disorder, or condition.

The terms “treating”, “treatment” or “alleviation” refer to both therapeutic treatment and prophylactic or preventative measures; wherein the object is to prevent, avoid the occurrence or slow down at least one adverse effect or symptom of the targeted pathologic condition or disease. Those in need of treatment include those already with the disease as well as those prone to have the disease or those in whom the disease is to be prevented. A subject or mammal is successfully “treated” for a disease or affection or condition if, after receiving the treatment according to the present invention, the subject or mammal shows observable and/or measurable reduction in or absence of one or more of the following: reduction ROS production; and/or relief to some extent, for one or more of the symptoms associated with the specific disease or condition; reduced morbidity and mortality, and improvement in quality of life issues. The above parameters for assessing successful treatment and improvement in the disease are readily measurable by routine procedures familiar to a physician.

The term “tympanic membrane” refers to the transparent, fibrous membrane that separates the external auditory canal from the eardrum body (middle ear cavity containing the ossicles) and transmits sound vibrations to the ossicles. The tympanic membrane consists of three superimposed layers: an outer skin layer, an intermediate fibrous layer and an inner mucous layer (on the side of the tympanic body). It is both strong and elastic. With a diameter of 9 to 10 millimetres, it fits into a groove in the bottom of the external auditory canal, the tympanic sulcus. The upper part of the eardrum is called the pars flaccida, and the lower part, to which the hammer handle (one of the ossicles) is attached, is called the pars tensa.

The term “tympanic area” refers to the surroundings of the “tympanic membrane”, on the side of the ear canal. Thus, the tympanic area encompasses the side of the tympanic membrane which regards the ear canal (i.e., the area of the ear canal that is proximal to the tympanic membrane) as well as the tympanic membrane itself. For example, for an adult human subject, the tympanic area may extend through the ear canal from the tympanic membrane towards the pinna in a distance of about 15 mm or less, about 10 mm or less or about 5 mm or less, wherein the distance is measured from the tympanic membrane along the ear canal.

### DETAILED DESCRIPTION

### Pharmaceutical Composition

This invention relates to a pharmaceutical composition comprising:
at least one active agent; and
a pharmaceutically acceptable vehicle,
   wherein the pharmaceutically acceptable vehicle comprises:
      at least one diffusing agent, and
      at least one solubilizing agent.

Typically, the pharmaceutical composition of the invention is for use in the treatment of an ear disorder and/or a neurological disorder.

Without willing to be linked by any theory, the Applicant suggests that the composition may act as a passive diffusion means following the ossicle with the help of the small artery feeding the ossicle. An advantage of the invention is that the pharmaceutical composition of the invention diffuses from the tympanic area to the inner ear, without perforation of the tympanic membrane.

Although some substances may possibly qualify simultaneously as an active agent, a diffusing agent and/or a solubilizing agent, in the pharmaceutical composition of the invention the active agent, the diffusing agent and the solubilizing agent refer to different ingredients, i.e., in the emulsion of the invention the “active agent” ingredient cannot at the same time be the “diffusing agent” ingredient nor a “solubilizing agent” ingredient, etc. In other words, the pharmaceutical composition of the invention systematically comprises at least three different ingredients, one of them performing at least the function of the active agent, another one of them performing at least the function of the diffusing agent, and the third ingredient performing at least the function of the solubilizing agent. In other words, in the pharmaceutical composition of the invention, the “active agent”, the “diffusing agent” and the “solubilizing agent” are each distinct substance from one another.

### Active Agent

The at least one active agent comprised in the pharmaceutical composition according to the invention may be any active agent suitable for preventing and/or treating at least one ear disorder and/or at least one neurological disorder, preferably at least one ear disorder, more preferably at least one inner ear disorder.

According to one embodiment, the at least one active agent is selected from the group consisting of repair agents, ionic channels antagonists, glutamate receptors antagonists, dopamine agonists or precursors, psychotropic drugs, antioxidant compounds, myorelaxant agents, vasodilators, diuretic compounds, other agents blocking signal transmission, nutritional supplements, anti-edema compounds, and any mixture thereof.

Repair agents are agents that are able to at least partially repair or restore the structure and/or functionality of body elements, especially ear elements, which dysfunction or alteration may contribute to at least one ear or neurological disease, such as cells, in particular ciliated cells, nerves, synapses and/or myelin. Repair agents may be selected from the group consisting of growth factors, such as neurotrophin-3 (NT3), neurotrophin-4 (NT4), nerve growth factor (NGF) or brain-derived neurotrophic factor (BDNF); growth factor up-regulating compounds; anti-regrowth antibodies, such as repulsive guidance molecule A (RGMa), neogenin or nogo-A protein; Tropomyosin Receptor Kinase (TRK) agonists, including Tropomyosin receptor kinase B (TrkB) (also known as tyrosine receptor kinase B, or BDNF/NT-3 growth factors receptor, or neurotrophic tyrosine kinase, receptor, type 2) agonists and Tropomyosin receptor kinase C (TrkC) (also known as NT-3 growth factor receptor, neurotrophic tyrosine kinase receptor type 3, or TrkC tyrosine kinase) agonists, such as 7,8-dihydroxyflavone (7,8-DHF), a 7,8-dihydroxyflavone bisphosphonate, a 7,8-dihydroxyflavone prodrug, deoxygedunin, gambogic acid, demethylasterriquinone B1 (DMAQ-B1), LM22B-10, LM22A-4 or TRK agonist antibodies; glycogen synthase 3 beta (GSK3β) inhibitors; histone deacetylase (HDAC) inhibitors; cJun N-terminal kinase ligands, such as AM-111; gene therapy agents, such as Atoh1; p27^{kip1} inhibitors; stem cells regenerating agents such as lithium, valproic acid or a salt thereof, for instance sodium valproate (Depakine®); and any mixture thereof.

According to one embodiment, the at least one active agent is a repair agent, wherein the repair agent is a Tropomyosin Receptor Kinase (TRK) agonist, preferably 7,8-dihydroxyflavone (7,8-DHF). According to one embodiment, the at least one active agent is a repair agent, wherein the repair agent is a growth factor, preferably neurotrophin-3 (NT3).

Ionic channels antagonists may be selected from the group consisting of sodium channels antagonists such as lidocaine, ambroxol (Surbronc®), carbamazepine (Tegretol®), ifenprodil (vadilex®), mexiletine, tetracaine or procaine; calcium channels antagonists such as nimodipine (nimotop®), verapamil or nicardipine (loxen®); potassium channels antagonists such as retigabine; and any mixture thereof.

Glutamate receptors antagonists may be selected from the group consisting of NMDA receptor antagonists such as gacyclidine, neramexane, caroverine, dextromethorphan (tussidane®), memantine, riluzole (rilutek®), ketamine, methadone, amantadine (mantadix®), ifenprodil (vadilex®), minocycline, atomoxetine, felbamate (taloxa®), esketamine (stravato®), acamprosate (aotal®), flupirtine, lanicemine, meperidine, haloperidol (haladol®), ebselen, ecstasy, magnesium or amantadine (mantadix®); AMPA receptor antagonists, such as perampanel or zinc; kainate receptor antagonists, such as selurampanel (BGG492); and any mixture thereof.

Dopamine agonists or precursors may be selected from the group consisting of dopamine and derivatives thereof, such as levodopa (L-DOPA) and/or carbidopa (sinemet®), or apomorphine; dopamine agonists increasing brain-derived neurotrophic factor BDNF such as rotigotine (neupro®), rasagiline (azilect®), benzerazide, or selegiline (deprenyl®); other dopamine agonists, such as ropinirole (adartrel®), sulpiride (dogmatil®), pramipexole (sifrol®), piribedil (trivastal®), ecstasy, cariprazine or amandatine (mantadix®); and any mixture thereof.

Psychotropic drugs may be selected from the group consisting of antidepressants increasing brain-derived neurotrophic factor BDNF such as amitriptyline (laroxyl®), clomipramine (anafranil®), imipramine (tofranil®), citalopram (seropram®), reboxetine, moclobemide (moclamine®) or fluoxetine (prozac®); other antidepressants, such as sertraline (zoloft®), nortriptyline (Aventyl®), paroxetine (deroxat®), deanxit, trazodone (trazolan®), bupropion (zyban®), venlafaxine (effexor®), rapastinel, desipramine, atomoxetine (strattera®), chlorpromazine (largactil®), tianeptine (stablon®), mianserin, chlorprothixene or terbutalin (bricanyl®); anti-epileptic and/or anti-convulsant agents, optionally acting on NMDA, GABA and/or acetylcholine, such as gabapentin (neurontin®), vigabatrin (sabril®), flunarizine, flumazenil (anexate®), gabamazepine, lamotrigine (lamictal®), pregabaline (lyrica®), oxcarbazepine (trileptal®), amino oxyacetic acid, retigabin (trobalt®), zonisamide (zonegran®), pregabaline (lyrica®), ethosuximide (zarontin®), trimethadione (tolvaptan®), topiramate (epitomax®), cannabidiol, or tianeptine; sleep improving agents, preferably of the benzodiazepine class, such as bromozepan (lexomil®), alprazolam (xanax®), clonazepam (rivotril®), midazolam, diphenhydramine (nautamine®), alimemazine (theralene®), or cyamemazine (tercian®); and any mixture thereof.

Antioxidant compounds may be selected from the group consisting of ebselen, flavonoids, such as isoquercetin, 7,8-dihydroxyflavone (7,8-DHF) or derivatives thereof, vitamin E and any mixture thereof.

Myorelaxant agents may be selected from the group consisting of cyclobenzaprine (flexeril®), cyclandelate, prenylamine, nilvadipine and any mixture thereof.

Vasodilators may be selected from the group consisting of trimetazidine (vastarel®), sildenafil (viagra®), diltiazem ifenprodil (vadilex®), and any mixture thereof.

Diuretic compounds may be for example furosemide.

Other agents blocking signal transmission may be selected from the group consisting of β2-adrenoreceptor agonists, such as formoterol, salbutamol (ventoline®), metaproterenol (also known as orciprenaline), procaterol, or clenbuterol; nicotine receptor antagonists, such as ondansetron (zophren®); NK1 receptor and/or substance P antagonists, such as rolapitant (varabi®) or aprepitant (emend®, cinvanti®); calcigenin gene related peptide (CGRP) inhibitors, such as ubrogepant (ubrelvy®) or rimegepant (nurtec®); sigma 1 receptor antagonists, such as BD1047; anti-histaminic agents, such as clemastine, hydroxyzine (atarax®), diphenhydramine (nautamine®), cinnarizine, terfenadine, alimemazine (theralene®), or cimetidine (tagamet®); neuropeptides, such as oxytocin; prostaglandin analogues, especially PGE1 analogues, such as misoprostol (cytotec®); opioids, such as naltrexone; glycinereceptor agonists, such as D-cycloserin (seromycin®); gamma aminobutyric acid GABA agonists, such as taurine; and any mixture thereof.

Nutritional supplements may be selected from the group consisting of vitamin E, TFA 12 vitamin E derivative, vitamin B2, vitamin B6, vitamin B12, vitamin C, melatonin, magnesium, zinc and any mixture thereof.

Anti-edema compounds may be selected from the group consisting of extranase, acetazolamide (diamox®), betahistine, dexamethasone and any mixture thereof.

In the present invention, the name of an active agent includes the active agent and any prodrug and/or salt thereof. Preferably, the name of an active agent includes the active agent and any salt thereof, preferably any pharmaceutically acceptable salt thereof.

According to a particular embodiment, the active agent is selected from the group consisting of 7,8-dihydroxyflavone, dexamethasone, dextromethorphan, lidocaine, gacyclidine, selurampanel, levodopa (L-DOPA), pramipexole, amitriptyline, cannabidiol CBD and ropinirole.

According to an embodiment, the at least one active agent is not ototoxic or not provided in the composition at a dose that would incur ototoxicity.

According to one embodiment, the pharmaceutical composition of the invention comprises one active agent. According to one embodiment, the pharmaceutical composition of the invention comprises a combination of at least two active agents, e.g., active agents as described hereinabove.

### Diffusing Agent

According to one embodiment, the diffusing agent is a terpene or a derivative thereof. In one embodiment, the terpene derivative is selected from terpene alcohols, terpene ketones, terpene aldehydes, and any mixture thereof.

In one embodiment, the terpene or the derivative thereof is selected from the group consisting of α-(-)-bisabolol ((2S)-6-methyl-2-[(1S)-4-methylcyclohex-3-en-1-yl]hept-5-en-2-ol, also known as levomenol), linalool (3,7-dimethylocta-1,6-dien-3-ol), limonene (1-methyl-4-(prop-1-en-2-yl)cyclohex-1-ene), and any mixture thereof.

In a one embodiment, the diffusing agent is a sesquiterpene alcohol. In a preferred embodiment, the diffusing agent is α-(-)-bisabolol or linalool. In a more preferred embodiment, the diffusing agent is α-(-)-bisabolol.

### Solubilizing Agent

A solubilizing agent is any compound suitable for solubilizing and/or dispersing, preferably solubilizing, at least part of the active agent(s) and/or diffusing agent(s). In an embodiment, the solubilizing agent is suitable for solubilizing the active agent(s). According to one embodiment, the solubilizing agent is selected from the group consisting of oils of medium or light viscosity, oil/water serum bases, glycerine, alcohols and any mixture thereof.

Oils of medium or light viscosity may be vegetable oils. Vegetable oils may for example be almond oil, apricot oil, arachis oil (peanut oil), camellia oil, castor oil, coconut oil (including fractionated coconut oil), cottonseed oil, evening primrose oil, grapeseed oil, jojoba oil, maize oil (corn oil), olive oil, rapeseed oil, rosehip oil, safflower oil, sesame oil, soybean oil, sunflower oil, medium chain triglycerides (MCT) (for example MCT sourced from Cocos nucifera or Elaeis guineensis), wheat germ oil, and any mixture thereof. According to one embodiment, the vegetable oils are pharmaceutically acceptable oils selected from the group consisting of almond oil, arachis oil, camellia oil, castor oil, coconut oil, cottonseed oil, maize oil, olive oil, rapeseed oil, safflower oil, sesame oil, soybean oil, sunflower oil, medium chain triglycerides (MCT), wheat germ oil, and any mixture thereof. According to one embodiment, the vegetable oils are selected from the group consisting of apricot oil, coconut oil (preferably fractionated coconut oil), evening primrose oil, grapeseed oil, jojoba oil, rosehip oil, and any mixture thereof. In one preferred embodiment, the vegetable oils are selected from the group consisting of apricot oil, fractionated coconut oil, evening primrose oil, grapeseed oil, jojoba oil and rosehip oil. In particular, the vegetable oil is apricot oil.

Oil/water serum bases may be for instance the oil/water serum base marketed by Fagron (Belgium, The Netherlands) under the trade name Seraqua™. Seraqua™ is an ultra-light O/W serum base comprising silicones.

Alcohols may be selected from the group consisting of propanediol, isopropanol, benzyl alcohol, ethanol, and any mixture thereof.

Preferably, the combination of the at least one active agent, the at least one diffusing agent and the at least one solubilizing agent is not ototoxic. One of ordinary skill in the art will be able to select, among the compounds listed for each category, the appropriate combinations of active agent(s), diffusing agent(s) and solubilizing agent(s) to be comprised in the pharmaceutical compositions in order to avoid any ototoxicity.

### Galenic Formulation

According to one embodiment, the pharmaceutical composition of the invention is formulated in formulations suitable for a local administration onto the tympanic membrane, such as semi-solid, gel, liquid, suspension, emulsion, osmotic dosage formulation, diffusion dosage formulation or erodible formulation.

In one embodiment, the pharmaceutical composition of the invention is formulated as a gel formulation, such as thermoreversible gel, hydrogel, glycerin-based gel, conjugated or crosslinked gel or alginate-based gel. In one preferred embodiment, the pharmaceutical composition of the invention is formulated as a liquid formulation, such as ear drops.

According to one embodiment, the pharmaceutical composition of the invention is formulated as a liquid dosage form.

According to one embodiment, the pharmaceutical acceptable vehicle of the pharmaceutical composition of the invention further contains conventional non-toxic pharmaceutically acceptable carriers and/or adjuvants appropriate for a local route of administration onto the tympanic membrane. The pharmaceutical acceptable vehicle of the pharmaceutical composition of the invention may optionally comprise one or more other pharmaceutically acceptable carriers. Such suitable carriers for use in the preparation of the administration forms will be clear to the skilled person; reference is made to the latest edition of Remington's Pharmaceutical Sciences. Especially, the pharmaceutically acceptable vehicle of the pharmaceutical composition of the invention can optionally contain such inactive substances that are commonly used in pharmaceutical formulations, such as for example cosolvents, antioxidants, surfactants, wetting agents, emulsifying agents, buffering agents, pH modifying agents, preservatives agents, stabilizers, stabilizing agents, precipitation inhibitors, lubricants, disintegrants, glidants, diluents, and the like.

“Gel formulation” refers to a semisolid system consisting of either a suspension made up of small inorganic particles or large organic molecules interpenetrated by a liquid. Gel formulation may consist as a single-phase or a two-phase system. A single-phase gel consists of organic macromolecules distributed uniformly throughout a liquid in such a manner that no apparent boundaries exist between the dispersed macromolecules and the liquid. Single-phase gels are usually prepared from synthetic macromolecules or from natural gums. In some embodiments, single-phase gels are generally aqueous, but will also be made using alcohols and oils. Two-phase gels consist of a network of small discrete particles. Gel formulation may be classified as hydrophobic or hydrophilic. The bases of a hydrophobic gel usually consist of a liquid paraffin with polyethylene or fatty oils gelled with colloidal silica, or aluminium or zinc soaps. In contrast, the bases of hydrophilic gels usually consist of water, glycerol, or propylene glycol gelled with a suitable gelling agent.

“Liquid formulation” refers to a pourable pharmaceutical formulation which contains a mixture of active agents and pharmaceutically acceptable ingredients (excipients) dissolved or suspended in a suitable solvent or mixtures of solvents. Liquid formulation can consist of a monophasic or a biphasic liquid dosage form. A monophasic liquid formulation is a one-phase system consisting of two components, the solute (the component that gets dissolved) and the solvent (the medium in which solute dissolves). A biphasic formulation may either be a suspension or an emulsion.

According to one embodiment, the pharmaceutically acceptable vehicle comprises, in addition to the diffusing agent(s) and the solubilizing agent(s), at least one carrier, solvent and/or co-solvent. Suitable carriers, solvent and/or co-solvents for the preparation of gel or liquid formulation may comprise purified or distilled water, alcohols such as ethanol, acetic acid, mineral oils, organic oily bases, propylene glycol, polyethylene glycol or any mixture thereof. According to one embodiment, the at least one solubilizing agent is the only carrier, solvent or co-solvent comprised in the vehicle.

According to one embodiment, suitable gelling agents for the preparation of gel formulation are selected from cellulose, cellulose derivatives (including hydroxypropylmethylcellulose, methylcellulose, hydroxypropylcellulo se, hydroxypropylmethylcellulose phthalate and hydroxypropylmethylcellulose acetate succinate), guar gum, xanthan gum, locust bean gum, alginates, such as alginic acid, silicates, starch, tragacanth, carboxyvinyl polymers, carragean, paraffin, petrolatum and a mixture thereof.

Some other ingredients may be added to the pharmaceutically acceptable vehicle of the pharmaceutical composition according to the invention. In some embodiments, surfactants are added, such as for example Cremophor RH40 (polyoxyl 40 hydrogenated castor oil), Cremophor EL (polyoxyl 35 hydrogenated castor oil), polysorbate 20 (polyoxyethylenesorbitan monolaurate), polysorbate 80 (polyoxyethylenesorbitan monooleate), poloxamer 188 (poly(ethylene glycol)-block-poly(propylene glycol)-block-poly(ethylene glycol)), poloxamer 407 (Poly(ethylene glycol)-block-poly(propylene glycol)-block-poly(ethylene glycol)), vitamin E TPGS (vitamin E polyethylene glycol succinate), Solutol HS15 (polyoxyethylated 12-hydroxystearic acid), Labrasol (caprylocaproyl polyoxyl-8 glycerides), Labrafil M1944 (Oleoyl polyoxyl-6 glycerides), polyvinylpyrrolidone (also called povidone, preferably polyvinylpyrrolidone K17, K19, K29-K32, K90), polyvinylpyrrolidone polyvinylacetate copolymer, polyvinyl caprolactam-polyvinyl acetate-polyethylene glycol graft copolymer (Soluplus®). polylactide polyethylene glycol block copolymer, carboxymethylcellulose (Na/Ca), polyethylene glycol methyl ether-block-poly(D-L-lactide) copolymer, sodium lauryl sulfate, sodium docusate, propylene glycol monolaurate, propylene glycol dilaurate, propylene glycol monocaprylate, polyethylene glycol 660 12-monostearate, poly(butyl methacrylate-co-(2-dimethylaminoethyl) methacrylate-co-methyl methacrylate) 1:2:1. In some embodiments, buffering agents are used to help to maintain the pH in the range that approximates physiological conditions. Suitable buffering agents include both organic and inorganic acids and salts thereof, such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used. In some embodiments, pH modifiers are added, such as for example sodium hydroxide, sodium bicarbonate, magnesium oxide, potassium hydroxide, meglumine, sodium carbonate, citric acid, tartaric acid, ascorbic acid, fumaric acid, succinic acid and malic acid. In some embodiments, preservatives agents are added to retard microbial growth. Suitable preservatives for use with the present disclosure include sodium bisulfate, sodium thiosulphate, ascorbate, phenol, benzyl alcohol, meta-cresol, methyl paraben, propyl paraben, octadecyldimethylbenzyl ammonium chloride, benzalkonium halides (e.g., chloride, bromide, and iodide), hexamethonium chloride, and alkyl parabens such as methyl or propyl paraben, catechol, resorcinol, cyclohexanol, and 3-pentanol. In some embodiments, stabilizers are added and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive which solubilizes the therapeutic agent or helps to prevent denaturation or adherence to the container wall or helps to inhibit the precipitation, particle growth or agglomeration of the active agent. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc.; organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thiosulfate; low molecular weight polypeptides (e.g., peptides of 10 residues or fewer); proteins such as human serum albumin, bovine serum albumin, gelatin or immunoglobulins; hydrophylic polymers, cellulose derivatives; monosaccharides, such as xylose, mannose, fructose, glucose; disaccharides such as lactose, maltose, sucrose and trisaccharides such as raffinose; polysaccharides such as dextran; polyethylene glycol methyl ether-block-poly(D-L-lactide) copolymer; poly(butyl methacrylate-co-(2-dimethylaminoethyl) methacrylate-co-methyl methacrylate) 1:2:1. In some embodiments, lubricants are added, such as for example magnesium stearate, glyceryl esters, behenoyl polyoxyl-8 glycerides Nf (Compritol HD5 ATO), sodium stearyl fumarate and the like. In some embodiments, viscosity enhancing agents are added, such as polyvinylpyrrolidone, polyvinyl alcohol, sodium chondroitin sulfate, sodium hyaluronate. Other suitable viscosity enhancing agents may include, but are not limited to, acacia (gum arabic), agar, aluminum magnesium silicate, sodium alginate, sodium stearate, bladderwrack, bentonite, carbomer, carrageenan, carbopol, xanthan, cellulose, microcrystalline cellulose (MCC), ceratonia, chitin, carboxyrnethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, sterculia gum, xanthan gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, carboxymethyl-cellulose (CMC), poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(nethoxyethoxyethyl methacrylate), sodium silicon dioxide, polyvinylpyrrolidone (PVP: povidone), Splenda(dextrose, maltodextrin and sucralose) or combinations thereof. In one embodiment, the viscosity-enhancing agent is a combination of carboxymethylated chitosan, or chitin, and alginate. In one embodiment, the combination of chitin and alginate with the at least one active agent as described hereinabove acts as a controlled release formulation, restricting the diffusion of the at least one active agent from the formulation. In one embodiment, the combination of carboxymethylated chitosan and alginate is optionally used to assist in increasing the transportation of the active ingredient towards inner ear. In some embodiments, emulsifying agents are added, such as for example carbomer, carrageenan, lanolin, lecithin, mineral oil, oleic acid, oleyl alcohol, pectin, poloxamer, polyoxyethylene sorbitan fatty acid esters, sorbitan esters, triethanolamine, propylene glycol monolaurate, propylene glycol dilaurate, propylene glycol monocaprylate.

The pharmaceutical compositions of the invention may conveniently be presented in dosage unit form and may be prepared by any of the methods well known in the art of pharmacy. All methods include the step of bringing the active agent into association with the carrier which constitutes one or more accessory ingredients. In general, the pharmaceutical compositions are prepared by uniformly and intimately bringing the at least one active agent into association with a liquid carrier or a finely divided solid carrier or both, and then, if necessary, shaping the product into the desired formulation. In the pharmaceutical composition, the active agent is preferably included in an amount sufficient to produce the desired effect upon the process or condition of diseases.

According to one embodiment, the pharmaceutical composition of the invention does not comprise hydrocortisone. According to one embodiment, the pharmaceutical composition of the invention does not comprise lactoferrin. According to one embodiment, the pharmaceutical composition of the invention does not comprise peroxidase. According to one embodiment, the pharmaceutical composition of the invention does not comprise squalene. According to one embodiment, the pharmaceutical composition of the invention does not comprise linalool. According to one embodiment, the pharmaceutical composition of the invention does not comprise panthenol. According to one embodiment, the pharmaceutical composition of the invention does not comprise allantoin. According to one embodiment, the pharmaceutical composition of the invention does not comprise tea tree oil. According to one embodiment, the pharmaceutical composition of the invention does not comprise squalane. According to one embodiment, the pharmaceutical composition of the invention does not comprise almond oil. According to one embodiment, the pharmaceutical composition of the invention does not comprise turmeric oil. According to one embodiment, the pharmaceutical composition of the invention does not comprise oregano oil. According to one embodiment, the pharmaceutical composition of the invention does not comprise ceftazidime. According to one embodiment, the pharmaceutical composition of the invention does not comprise eucalyptus oil. According to one embodiment, the pharmaceutical composition of the invention does not comprise petrolatum. According to one embodiment, the pharmaceutical composition of the invention does not comprise cannabidiol (CBD). According to one embodiment, the pharmaceutical composition of the invention does not comprise gacyclidine. According to one embodiment, the pharmaceutical composition of the invention does not comprise pramipexol.

### Amount of Ingredients

According to one embodiment, the pharmaceutical composition of the invention comprises a therapeutically effective amount of the at least one active agent as described hereinabove. The therapeutically effective amount of the at least one active agent can be determined by a person skilled in the art based on his general knowledge, the pharmacokinetic parameters of the at least one active agent, the subject's age, health condition, etc.

According to one embodiment, the at least one active agent is at a concentration ranging from 0.001% to 20%, preferably ranging from 0.01% to 10%, more preferably ranging from 0.1% to 5%, furthermore preferably ranging from 1% to 2.5%, by weight based on the total weight of the composition (w/w). In one embodiment, the at least one active agent is at a concentration ranging from 1% to 20% by weight based on the total weight of the composition (w/w). In one embodiment, the at least one active agent is at a concentration ranging from 2% to 10% by weight based on the total weight of the composition (w/w). In one embodiment, the at least one active agent is at a concentration of about 2% w/w, about 3% w/w, about 4% w/w, about 5% w/w, about 6% w/w, about 7% w/w, about 8% w/w, about 9% w/w or about 10% by weight based on the total weight of the composition (w/w). In a preferred embodiment, the at least one active agent is at a concentration of about 5% by weight based on the total weight of the composition (w/w).

According to one embodiment, the at least one diffusing agent, e.g., α-(-)-bisabolol or linalool, preferably α-(-)-bisabolol, is at a concentration ranging from 0.1% to 10%, preferably ranging from 0.1% to 5%, by weight based on the total weight of the composition (w/w). In one embodiment, the at least one diffusing agent is at a concentration ranging from 0.5% to 5% by weight based on the total weight of the composition (w/w). In one embodiment, the at least one diffusing agent is at a concentration of about 0.5% w/w, about 1% w/w, about 2% w/w, about 3% w/w, about 4% w/w or about 5% by weight based on the total weight of the composition (w/w). In a preferred embodiment, the at least one diffusing agent is at a concentration of about 1% by weight based on the total weight of the composition (w/w).

According to one embodiment, the pharmaceutical composition of the invention comprises a stabilizer, preferably mannitol, at a concentration ranging from 0.5% to 10% by weight based on the total weight of the composition (w/w). In one embodiment, the stabilizer is at a concentration ranging from 1% to 10% by weight based on the total weight of the composition (w/w). In one embodiment, the stabilizer is at a concentration of about 2% w/w, about 3% w/w, about 4% w/w, about 5% w/w, about 6% w/w or about 7% by weight based on the total weight of the composition (w/w). In a preferred embodiment, the stabilizer is at a concentration of about 5.8% by weight based on the total weight of the composition (w/w). According to one embodiment, the pharmaceutical composition of the invention comprises at least one vehicle, solvent or co-solvent at a concentration ranging from 40% to 98% by weight based on the total weight of the composition (w/w). In one embodiment, the at least one vehicle, solvent or co-solvent is at a concentration ranging from 50% to 98% by weight based on the total weight of the composition (w/w). In one embodiment, the at least one vehicle, solvent or co-solvent is at a concentration ranging from 80% to 98% by weight based on the total weight of the composition (w/w). In one embodiment, the at least one vehicle, solvent or co-solvent is at a concentration of about 80% w/w, about 81% w/w, about 82% w/w, about 83% w/w, about 84% w/w, about 85% w/w, about 86% w/w, about 87% w/w, about 88%, w/w about 89% w/w, about 90% w/w, about 91% w/w, about 92% w/w, about 93% w/w, about 94% w/w, about 95% w/w, about 96% w/w, about 97% w/w or about 98% w/w by weight based on the total weight of the composition (w/w). In a preferred embodiment, the at least one preservative agent is at a concentration of about 95% by weight based on the total weight of the composition (w/w). According to one embodiment, the pharmaceutical composition of the invention further comprises at least one preservative agent at a concentration ranging from 0.5% to 10% by weight based on the total weight of the composition (w/w). In one embodiment, the at least one preservative agent is at a concentration ranging from 0.5% to 5% by weight based on the total weight of the composition (w/w). In one embodiment, the at least one preservative agent is at a concentration of about 0.5% w/w, about 1% w/w, about 2% w/w, about 3% w/w, about 4% w/w or about 5% by weight based on the total weight of the composition (w/w). In a preferred embodiment, the at least one preservative agent I sat a concentration of about 1% by weight based on the total weight of the composition (w/w). According to one embodiment, the pharmaceutical composition of the invention further comprises at least one diluent at a concentration ranging from 1% to 20% by weight based on the total weight of the composition (w/w). In one embodiment, the at least one diluent is at a concentration ranging from 2% to 10% by weight based on the total weight of the composition (w/w). In one embodiment, the at least one diluent is at a concentration of about 2% w/w, about 3% w/w, about 4% w/w, about 5% w/w, about 6% w/w, about 7% w/w, about 8% w/w, about 9% w/w or about 10% by weight based on the total weight of the composition (w/w). In a preferred embodiment, the at least one diluent is at a concentration of about 6% by weight based on the total weight of the composition (w/w).

In one embodiment, the pharmaceutical composition of the invention comprises: a) from 1% to 20% w/w of at least one active agent; preferably from 2% to 10% w/w, more preferably of about 2% w/w, about 3% w/w, about 4% w/w, about 5% w/w, about 6% w/w, about 7% w/w, about 8% w/w, about 9% w/w or about 10%, more preferably from 4% to 6% w/w, more preferably about 5% w/w; b) from 0.1% to 10% w/w of at least one diffusing agent, preferably from 0.1% to 5% w/w, preferably of about 0.5% w/w, about 1% w/w, about 2% w/w, about 3% w/w, about 4% w/w or about 5% w/w, more preferably of about 1% w/w; c) from 40% to 98% w/w of at least one vehicle, solvent or co-solvent, preferably from 80% to 98% w/w, more preferably of about 80% w/w, about 81% w/w, about 82% w/w, about 83% w/w, about 84% w/w, about 85% w/w, about 86% w/w, about 87% w/w, about 88% w/w, about 89% w/w, about 90% w/w, about 91% w/w, about 92% w/w, about 93% w/w, about 94% w/w, about 95% w/w, about 96% w/w, about 97% w/w or about 98% w/w, even more preferably of about 95% w/w; d) from 0.5% to 10% w/w of at least one preservative agent, preferably from 0.5% to 5% w/w, preferably from of about 0.5% w/w, about 1% w/w, about 2% w/w, about 3% w/w, about 4% w/w or about 5% w/w, more preferably of about 1% w/w; and e) from 1% to 20% w/w of at least one diluent; preferably from 2% to 10% w/w, more preferably of about 2% w/w, about 3% w/w, about 4% w/w, about 5% w/w, about 6% w/w, about 7% w/w, about 8% w/w, about 9% w/w or about 10%, more preferably from 4% to 6% w/w, more preferably about 5% w/w; by weight based on the total weight of the composition (w/w).

### Methods of Administration

According to one embodiment, the pharmaceutical composition of the invention is for a local administration in the ear canal (i.e., into the ear canal or onto the tympanic membrane). In one embodiment, the pharmaceutical composition is for administration into the ear canal. In one embodiment, the pharmaceutical composition is for administration onto the tympanic membrane.

This invention therefore encompasses a method for administering an active principle as described herein in the ear canal, for the treatment of inner ear disorder or neurological disorders. According to one embodiment, the pharmaceutical composition of the invention is administered in the ear canal. In one embodiment, the pharmaceutical composition is administered in the tympanic area. According to one embodiment, the administration does not include a step of injection through the tympanic membrane. In one embodiment, the administration does not include any invasive step, i.e., the administration is “non-invasive”.

Advantageously, the pharmaceutical composition of the invention is, through local means of administration in the ear canal, distributed into the inner ear. According to one embodiment, the at least one active agent as described hereinabove is observed into the tympanic bulla and into the perilymph at a concentration ranging from 100 ng/ml to 150000 ng/ml. In one embodiment, the at least one active agent as described hereinabove is observed into the tympanic bulla and into the perilymph at a concentration ranging from 100 ng/ml to 500 ng/ml; or ranging from 500 ng/ml to 1000 ng/ml; or ranging from 1000 ng/ml to 5000 ng/ml; or ranging from 5000 ng/ml to 10000 ng/ml; or ranging from 10000 ng/ml to 20000 ng/ml; or ranging from 20000 ng/ml to 30000 ng/ml; or ranging from 30000 ng/ml to 40000 ng/ml; or ranging from 40000 ng/ml to 50000 ng/ml; or ranging from 50000 ng/ml to 60000 ng/ml; or ranging from 60000 ng/ml to 70000 ng/ml; or ranging from 70000 ng/ml to 100000 ng/ml; or ranging from 100000 ng/ml to 125000 ng/ml; or ranging from 125000 ng/ml to 150000 ng/ml.

### Dose

Depending on the condition to be prevented or treated and the form of administration, the pharmaceutical composition of the invention may be administered as a single daily dose, or divided over one or more daily doses. In one embodiment, the pharmaceutical composition of the invention is administered at a dose such that it corresponds to administering about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5 to 5 or 5 to 50 mg/kg per day. The pharmaceutical composition of the invention may be administered on a regimen of one to 4 times per day, preferably once or twice per day. It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### Medical Use and Methods of Treatment

According to one embodiment, the pharmaceutical composition of the invention, as described hereinabove, is for use in the treatment of an ear disorder and/or a neurological disorder. According to one preferred embodiment, the pharmaceutical composition is for use in the treatment of an ear disorder.

In one embodiment, the ear disorder is an inner ear disorder. Non-limiting example of inner ear disorders include otosclerosis; nonobliterative and obliterative otosclerosis involving oval window; cochlear otosclerosis; acoustic neuroma; bilateral and unilateral sensorineural hearing loss such as noise-induced hearing loss or aged-induced hearing loss such as presbycusis; bilateral and unilateral conductive hearing loss; bilateral and unilateral mixed conductive and sensorineural hearing loss; sudden idiopathic hearing loss; drug-induced hearing loss; single sided deafness; ear infection; ototoxicity; drug-induced ototoxicity; herpes zoster oticus; labyrinthitis; purulent labyrinthitis; Labyrinthine fistula; Labyrinthine dysfunction; vestibular neuronitis; excitotoxicity; autoimmune inner ear disease; acute unilateral vestibulopathy; vestibular neuronitis; benign paroxysmal positional vertigo; vertigo of central origin; Meniere's disease; Meniere's syndrome and tinnitus. According to a preferred embodiment, the pharmaceutical composition of the invention is for use in the treatment of hearing loss, e.g., bilateral and unilateral sensorineural hearing loss such as noise-induced hearing loss or aged-induced hearing loss such as presbycusis; bilateral and unilateral conductive hearing loss; bilateral and unilateral mixed conductive and sensorineural hearing loss; sudden idiopathic hearing loss; drug-induced hearing loss; or single sided deafness. According to a preferred embodiment, the pharmaceutical composition of the invention is for use in the treatment of tinnitus.

According to one embodiment, the active agent is a dopamine agonist and the pharmaceutical composition is for use in the treatment of tinnitus. According to one embodiment, the active agent is a dopamine agonist and the pharmaceutical composition is for use in the treatment of vertigo. According to one embodiment, the active agent is a dopamine agonist and the pharmaceutical composition is for use in the treatment of Meniere's disease. According to one embodiment, the active agent is a dopamine precursor, wherein the dopamine precursor is levodopa (L-DOPA), and the pharmaceutical composition is for use in the treatment of vestibular disorders.

According to another embodiment, the pharmaceutical composition of the invention, as described hereinabove, is for use in the treatment of a neurological disorder. In one embodiment, the neurological disorder is selected from a mental and behavioral disorder or a disease of the nervous system.

Without being linked by any theory, the Applicant suggests that the pharmaceutical composition for use according to the invention may be able to diffuse directly into the inner ear and to reach the brain through the vascularization.

In one embodiment, the neurological disorder is a mental and behavioural disorder. Non-limiting examples of mental and behavioural disorders include dementia in Alzheimer's disease; vascular dementia; organic amnesic syndrome; delirium; mental disorders due to brain damage; mental and behavioural disorders due to psychoactive substance use; schizophrenia; schizotypal disorder; delusional disorder; acute and transient psychotic disorders; schizoaffective disorders; mood affective disorders such as manic episode, bipolar affective disorder, depressive episode, recurrent depressive disorder, persistent affective disorder; Neurotic, stress-related and somatoform disorders such as phobic anxiety disorders, obsessive-compulsive disorder, dissociative disorder; somatoform disorder; Behavioural syndromes associated with physiological disturbances and physical factors such as eating disorder, nonorganic sleep disorder, sexual dysfunction; Disorders of adult personality and behaviour such as specific personality disorders; Disorders of psychological development; Behavioural and emotional disorders with onset usually occurring in childhood and adolescence.

In one embodiment, the neurological disorder is a disease of the nervous system. Non-limiting examples of diseases of the nervous system include inflammatory diseases of the central nervous system, such as bacterial meningitis, meningitis in bacterial diseases, meningitis in infectious diseases, encephalitis, myelitis, encephalomyelitis, intracranial and intraspinal abscess and granuloma, intracranial and intraspinal phlebitis and thrombophlebitis and sequelae of inflammatory diseases of central nervous system; systemic atrophies primarily affecting the central nervous system, such as Huntington disease, hereditary ataxia, spinal muscular atrophy and related syndromes, systemic atrophies primarily affecting central nervous system and post-polio syndrome; Extrapyramidal and movement disorders such as Parkinson disease; Other degenerative diseases of the nervous system such as Alzheimer disease; Demyelinating diseases of the central nervous system such as multiple sclerosis, acute disseminated demyelination; Episodic and paroxysmal disorders such as epilepsy, status epilepticus, migraine, transient cerebral ischaemic attacks and related syndromes, vascular syndromes of brain in cerebrovascular diseases; Polyneuropathies and other disorders of the peripheral nervous system such as hereditary and idiopathic neuropathy, inflammatory polyneuropathy and Diseases of myoneural junction and muscle.

According to one embodiment, the active agent is a dopamine agonist and the pharmaceutical composition is for use in the treatment of neurological disorders, especially fatigue, restless leg syndrome or Parkinson's disease. According to one embodiment, the active agent is a dopamine agonist, wherein the dopamine agonist is cariprazine and the pharmaceutical composition is for use in the treatment of neuropsychiatric disorders, for example schizophrenia. According to one embodiment, the active agent is a dopamine precursor, wherein the dopamine precursor is levodopa (L-DOPA), and the pharmaceutical composition is for use in the treatment of Parkinson's disease.

The pharmaceutical composition for use of the invention may be used in monotherapy or in combination therapy in a subject in need of therapeutic and/or preventive treatment. Thus, according to a first embodiment, the pharmaceutical composition for use of the invention is administered to the subject without any other active agent. Thus, according to a second embodiment, the pharmaceutical composition for use of the invention is administered to the subject in combination with at least one additional active agent, e.g., an active agent as described hereinabove, optionally also formulated in a pharmaceutical composition according to the present invention. In one embodiment, the pharmaceutical composition is administered to the subject sequentially, simultaneously and/or separately with the other active agent as described hereinabove. According to one embodiment, the pharmaceutical composition for use of the invention comprises one active agent. According to one embodiment, the pharmaceutical composition for use of the invention comprises a combination of at least two active agents, e.g., active agents as described hereinabove.

Preferably, the subject in need of therapeutic and/or preventive treatment is a warm-blooded animal, more preferably a human According to one embodiment, the subject is a male. According to one embodiment, the subject is a female. According to one embodiment, the subject is an adult, i.e., over 18 years of age. According to one embodiment, the subject is a child, i.e., under 18 years of age. According to one embodiment, the subject is an infant, i.e., having an age of more than one month and less than two years. According to one embodiment, the subject is a newborn, i.e., having an age from birth to less than one month. According to a preferred embodiment, the subject is of greater than 50, 55, 60, 65, 70, 75, 80, 85, 90 or 95 years of age. In one embodiment, the subject is of greater than 65, 70, 75, 80, 85, 90 or 95 years of age. According to another preferred embodiment, the subject is of less than 20, 15, 10 or 5 years of age. In one embodiment, the subject is of less than 18, 17, 16, 15,14, 13, 12, 11, 10, 9, 8, 7, 6, 5, 4, 3 or 2 years of ages.

According to one embodiment, the subject does not suffer from any underlying pathology.

According to one embodiment, the subject is at risk of developing an ear disorder as described above. According to one embodiment, the subject is suffering from at least one risk factor, i.e., a pre-existing disease, condition, habit or behavior that may lead to an increased risk of developing an ear disorder. In one embodiment, the subject presents at least one risk factor selected from genetic causes, complications at birth, certain infectious diseases, chronic ear infections, the use of particular drugs, exposure to excessive noise, ageing, injury to the head, genetic predisposition, autoimmune diseases exposure to passive smoking (also referred to as environmental exposure smoking), patient below 4 years of age, patient above 65 years of age.

According to one embodiment, the pharmaceutical composition for use of the invention is administered to the subject at a volume ranging from 5 μl to 100 μl per ear. In one embodiment, the pharmaceutical composition for use of the invention is administered to the subject at a volume ranging from 10 μl to 50 μl per ear. In one embodiment, the pharmaceutical composition for use of the invention is administered to the subject at a volume ranging from 15 μl to 40 μl per ear.

According to one embodiment, the subject in need of therapeutic and/or preventive treatment is diagnosed by a health professional. For example, ear disorder is diagnosed by any examination routinely carried out in the medical setting, including tympanometry, acoustic reflectometry, hearing test or tympanocentesis.

By way of example, causes of tinnitus may be assessed based on the sound the subject hears, as follows: (i) Clicking: Muscle contractions in and around the ear can cause sharp clicking sounds that the subject hears in bursts. They may last from several seconds to a few minutes; (ii) Rushing or humming These sound fluctuations are usually vascular in origin, and may be notice when exercising or changing positions, such as lying down or standing up; (iii) Heartbeat: Blood vessel problems, such as high blood pressure, an aneurysm or a tumor, and blockage of the ear canal or eustachian tube can amplify the sound of the heartbeat in the ears (pulsatile tinnitus); (iv) Low-pitched ringing: Conditions that can cause low-pitched ringing in one ear include Meniere's disease. Tinnitus may become very loud before an attack of vertigo—a sense that the subject or his surroundings are spinning or moving; (v) High-pitched ringing: Exposure to a very loud noise or a blow to the ear can cause a high-pitched ringing or buzzing that usually goes away after a few hours. However, if there's hearing loss as well, tinnitus may be permanent. Long-term noise exposure, age-related hearing loss or medications can cause a continuous, high-pitched ringing in both ears. Acoustic neuroma can cause continuous, high-pitched ringing in one ear; (vi) Other sounds: Stiff inner ear bones (otosclerosis) can cause low-pitched tinnitus that may be continuous or may come and go. Earwax, foreign bodies or hairs in the ear canal can rub against the eardrum, causing a variety of sounds.

According to one embodiment, the medical use of the invention comprises a step of administering the pharmaceutical composition of the invention in the ear canal. In one embodiment, the medical use comprises a step of administering the pharmaceutical composition in the tympanic area. According to one embodiment, the medical use of the invention does not include a step of injection through the tympanic membrane. In one embodiment, the medical use does not include any invasive step, i.e., the medical use is “non-invasive”.

This invention also relates to the use of a pharmaceutical composition of the invention, as described hereinabove, in the treatment of an ear disorder and/or a neurological disorder as described hereinabove. This invention also relates to the use of a pharmaceutical composition of the invention, as described hereinabove, in the manufacture of a medicament for the treatment of an ear disorder and/or a neurological disorder as described hereinabove.

This invention also relates to a method for the treatment of an ear disorder and/or a neurological disorder, as described hereinabove, in a subject in need thereof, comprising a step of administrating to the subject a therapeutically effective dose of a pharmaceutical composition of the invention, as described hereinabove.

### Device

This invention also relates to a device for the local administration of the pharmaceutical composition of the invention, as described hereinabove, in the ear canal or onto the tympanic membrane via the ear canal. According to one embodiment, the device of the invention comprises a pump, a reservoir containing the pharmaceutical composition of the invention, and a catheter channel for the local administration of the pharmaceutical composition onto the tympanic membrane via the ear canal.

### EXAMPLES

The present invention is further illustrated by the following examples.

### Example 1: Pharmacokinetics Study of Different Active Agents

Pharmacokinetics study of different active agents has been conducted with ready-to-use formulations. The pharmacokinetic study consists of a single administration up to the eardrum on male Long Evans rats and assessment of the level of the active agent in middle ear area (tympanic bulla) and in the inner ear area (perilymph). Three formulations containing lidocaine, one formulation containing gacyclidine, two formulations containing 7,8-dihydroxyflavone (7,8-DHF), one formulation containing pramipexole, one formulation containing levodopa (L-DOPA), one formulation containing dextromethorphan and one formulation containing selurampanel were assessed in this study.

The concentration of lidocaine was quantified in the tympanic bulla and in the perilymph at 3 timepoints (1, 4 and 8 hours) for the two formulations. The concentration of gacyclidine, 7,8-dihydroxyflavone, pramipexole, levodopa, dextromethorphan and selurampanel was quantified in the perilymph at 3 timepoints (1, 4 and 8 hours).

### I. Materials and Methods

### 1. Formulations

The formulations are ready-to-use formulations and are according to Table 1 below:

TABLE 1

| Formulation | 1 | 2 | 3 | 4 | 5 |
|---|---|---|---|---|---|
| lidocaine HCl | 5% | 5% | 5% | — | — |
| gacyclidine | — | — | — | 5% | — |
| 7,8-dihydroxyflavone | — | — | — | — | 5% |
| pramipexole di-HCl | — | — | — | — | — |
| levodopa | — | — | — | — | — |
| dextromethorphan | — | — | — | — | — |
| selurampanel | — | — | — | — | — |
| amitriptyline | — | — | — | — | — |
| dexamethasone | — | — | — | — | — |
| cannabidiol | — | — | — | — | — |
| ropinirole | — | — | — | — | — |
| α-(−)-bisabolol | 1% | 1% | 1% | 1% | 1% |
| benzyl alcohol | 1% | 1% | 1% | 1% | 1% |
| isopropanol | — | — | — | — | — |
| jojoba oil | | | | | |
| mannitol | — | 5.8% | — | — | — |
| glycerine | qs for | qs for | — | qs for | qs for |
| | 1 vial | 1 vial | | 1 vial | 1 vial |
| Seraqua ™ | — | — | qs for | — | — |
| | | | 1 vial | | |
| distilled water | — | — | — | — | — |

| Formulation | 6 | 7 | 8 | 9 | 10 |
|---|---|---|---|---|---|
| lidocaine HCl | — | — | — | — | — |
| gacyclidine | — | — | — | — | — |
| 7,8-dihydroxyflavone | 5% | — | — | — | — |
| pramipexole di-HCl | — | 5% | — | — | — |
| levodopa | — | — | 5% | — | — |
| dextromethorphan | — | — | — | 5% | — |
| selurampanel | — | — | — | — | 5% |
| amitriptyline | — | — | — | — | — |
| dexamethasone | — | — | — | — | — |
| cannabidiol | — | — | — | — | — |
| ropinirole | — | — | — | — | — |
| α-(−)-bisabolol | 1% | 1% | 1% | 1% | 1% |
| benzyl alcohol | 1% | 1% | 1% | 1% | 1% |
| isopropanol | — | — | — | — | — |
| jojoba oil | | | | | |
| mannitol | — | — | — | — | — |
| glycerine | — | qs for | qs for | qs for | qs for |
| | | 1 vial | 1 vial | 1 vial | 1 vial |
| Seraqua ™ | qs for | — | — | — | — |
| | 1 vial | | | | |
| distilled water | — | — | — | — | — |

| Formulation | 11 | 12 | 13 | 14 | 15 | 16 |
|---|---|---|---|---|---|---|
| lidocaine HCl | — | — | — | — | 5% | — |
| gacyclidine | — | — | — | — | — | — |
| 7,8-dihydroxyflavone | — | — | — | — | — | 5% |
| pramipexole di-HCl | — | — | — | — | — | — |
| levodopa | — | — | — | — | — | — |
| dextromethorphan | — | — | — | — | — | — |
| selurampanel | — | — | — | — | — | — |
| amitriptyline | 5% | — | — | — | — | — |
| dexamethasone | — | 5% | — | — | — | — |
| cannabidiol | — | — | 5% | — | — | — |
| ropinirole | — | — | — | 5% | — | — |
| α-(−)-bisabolol | 1% | 1% | 1% | 1% | 1% | 1% |
| benzyl alcohol | 1% | 1% | 1% | 1% | 1% | 1% |
| isopropanol | — | — | — | — | 64% | 64% |
| jojoba oil | | | 20% | | | |
| mannitol | — | 5.8% | — | — | — | — |
| glycerine | qs for | qs for | qs for | qs for | — | — |
| | 1 vial | 1 vial | 1 vial | 1 vial | | |
| Seraqua ™ | — | — | — | — | — | — |
| distilled water | — | — | — | — | qs for | qs for |
| | | | | | 1 vial | 1 vial |

The amounts are indicated in weight relative to the total weight of the formulation. The formulations are stored at room temperature.

2. Route of administration: 20 μl of each formulation are delivered in the ear canal of one animal in one administration.

3. Animals: 6 male rats Long Evans, were obtained from Janvier Labs, Le Genest St Isle, 53941 St Berthevin, France. Acclimation Animals arrived on site at least 5 days before the experiment to allow optimal acclimation. Selection Animal health was examined on the day before starting the study, to ensure that only animals in good health were selected to enter the testing procedures. Identification: At the beginning of the study, animals were identified with a number on the tail. Each cage was labelled with a record card, indicating the test compound, the dose of administration and the identification of the animals Housing: Rats were housed in cages (1354G (Tecniplast, Lyon), dim 595*380 mm (w*d)), in a room where the air was continuously filtered, thereby avoiding any contamination. During experiments, rats were caged in groups of five (300-400 g) at a constant temperature with a day/night cycle of 12/12 hours. Animals received water (autoclaved water) and nutrition (ref A04C10), ad libitum.

4. Treatment and study design: The treatment was delivered into the ear canal and on the tympanic membrane on anesthetized rats (2.5% isoflurane). The exact volume of administration was determined during this first test and delivered (volume of 20 μL). Both ears were treated. After administration, the animal was kept on a heating pad for 5 minutes, positioned on its side to allow the compound to diffuse through the tympanic membrane. Then the second ear were administered, and the animal kept on its side for 5 minutes. Finally, the animal was kept on a ventral position until its recovery from anesthesia. The diffusion trough the tympanic membrane and into the inner ear and was assessed with the dosage of the perilymph sampling. Tympanic bulla sampling technique: After sacrifice of the rats, the right tympanic bulla was collected from each animal and transferred to an Eppendorf tube, weighed individually and frozen at −20° C. until analysis. Perilymph sampling technique: After sacrifice of the rats and extraction of the tympanic bulla from the left ear, a volume of about 1 μL of cochlear fluid at the base of the cochlea was sampled ex vivo. Tubes were weighed individually before and after sampling, and immediately transferred to a −20° C. freezer.

5. Bioanalysis: The concentration of each compound in the perilymph and in the tympanic bulla was quantified using an exploratory LC/MS-MS assay method in outsourcing laboratory (Eurofins Vergeze, France). The concentration of amitriptyline in plasma was assessed.

### II. Results

Study results are reported in Tables 2 and 3 below. “B.L.” means “Below the limit of quantification”

TABLE 2

| | | | Content (ng/g) | Concentration |
|---|---|---|---|---|
| | Time of | | in tympanic | (ng/mL) in |
| Formulation | sampling | | bulla | perilymph |
| 1 | 1 | hour | 505880 | 10189 |
| | 4 | hours | 54204 | 2259 |
| | 8 | hours | 529.2 | 1397 |
| 2 | 1 | hour | 138036 | 5542.4 |
| | 4 | hours | 7894.4 | 1535.4 |
| | 8 | hours | 509.96 | 1562.9 |
| 3 | 1 | hour | — | 16400 |
| | 4 | hours | — | 8220 |
| | 8 | hours | — | 229 |
| 4 | 1 | hour | — | 405.87 |
| | 4 | hours | — | 211.71 |
| | 8 | hours | — | B.L. |
| 5 | 1 | hour | — | 509.64 |
| | 4 | hours | — | 57.072 |
| | 8 | hours | — | 274 |
| 6 | 1 | hour | — | 1220 |
| | 4 | hours | — | 306 |
| | 8 | hours | — | 73.6 |
| 7 | 1 | hour | — | 39.097 |
| | 4 | hours | — | 2434.2 |
| | 8 | hours | — | 25.683 |
| 8 | 1 | hour | — | 9250.7 |
| | 4 | hours | — | 2593.5 |
| | 8 | hours | — | 3781.9 |
| 9 | 1 | hour | — | 381 |
| | 4 | hours | — | B.L. |
| | 8 | hours | — | 47.4 |
| 10 | 1 | hour | — | 2380 |
| | 4 | hours | — | 189 |
| | 8 | hours | — | 836 |
| 11 | 1 | hour | — | 469.91 |
| | 4 | hours | — | 36.876 |
| | 8 | hours | — | 21.635 |
| 12 | 1 | hour | — | 17.2 |
| | 4 | hours | — | 144 |
| | 8 | hours | — | 2360 |
| 13 | 1 | hour | — | 24.7 |
| | 4 | hours | — | 4030 |
| | 8 | hours | — | 4890 |
| 14 | 1 | hour | — | 123 |
| | 4 | hours | — | 1600 |
| | 8 | hours | — | B.L. |
| 15 | 1 | hour | — | 1370 |
| | 4 | hours | — | 505 |
| | 8 | hours | — | B.L. |
| 16 | 1 | hour | — | 296 |
| | 4 | hours | — | 92.9 |
| | 8 | hours | — | 60.6 |

TABLE 3

| | | |
|---|---|---|
| | | Concentration |
| | Time of | (ng/mL) |
| Formulation | Sampling | in plasma |

| | | | |
|---|---|---|---|
| 11 | 1 | hour | 65.101 |
| | 4 | hours | 9.4371 |
| | 8 | hours | 6.5471 |

### III. Conclusion

a) Presence in perilymph: Table 2 shows that the active agents: lidocaine, gacyclidine, 7,8-dihydroxyflavone, pramipexole, levodopa, dextromethorphan, selurampanel, amitriptyline, dexamethasone, cannabidiol and ropinirole are present within the inner ear (perilymph) after 8 hours from the administration onto the tympanic membrane. This study on 16 formulations highlights the ability of the pharmaceutical formulation of the invention to distribute the active agent into the inner ear after a local administration onto the tympanic membrane.

b) Presence in plasma: Table 3 shows that the active agent amitriptyline is present in plasma from 1 to 8 hours after administration. The concentration of amitriptyline in plasma decreases from 1 to 8 hours, which correlates with the results observed in perilymph.

### Example 2: Comparison of Solubilizing Agents

The formulations are ready-to-use formulations and are according to Table 1 above and Table 4 below:

TABLE 4

| Formulation | 17 | 18 | 19 | 20 |
|---|---|---|---|---|
| levodopa | — | — | 5% | 5% |
| dexamethasone | 5% | 5% | — | — |
| α-(−)-bisabolol | 1% | 1% | 1% | 1% |
| benzyl alcohol | 1% | 1% | 1% | 1% |
| isopropanol | — | 60% | — | 60% |
| Seraqua ™ | qs for | — | qs for | — |
| | 1 vial | | 1 vial | |
| distilled water | — | qs for | — | qs for |
| | | 1 vial | | 1 vial |

The same materials and methods were used as in Example 1. Results are presented in Table 5 below.

TABLE 5

| | | | Content (ng/g) | Concentration |
|---|---|---|---|---|
| | Time of | | in tympanic | (ng/mL) in |
| Formulation | sampling | | bulla | perilymph |
| 17 | 1 | hour | — | 503 |
| | 4 | hours | — | 797 |
| | 8 | hours | — | 525 |
| 18 | 1 | hour | — | 213 |
| | 4 | hours | — | 48.9 |
| | 8 | hours | — | 683 |
| 19 | 1 | hour | — | 1130 |
| | 4 | hours | — | 421 |
| | 8 | hours | — | 34.1 |
| 20 | 1 | hour | — | 52 |
| | 4 | hours | — | 104 |
| | 8 | hours | — | 42.1 |

These results are combined to those obtained above for the following compositions:
composition 12 (dexamethasone in glycerine),
composition 8 (levodopa in glycerine),
composition 5 (7,8-DHF in glycerine),
composition 6 (7,8-DHF in Seraqua™)
composition 16 (7,8-DHF in isopropanol),
composition 1 (lidocaine in glycerin),
composition 3 (lidocaine in Seraqua™), and
composition 15 (lidocaine in isopropanol).

The combined results show that, for all active agents (levodopa, dexamethasone, 7,8-DHF and lidocaine), any one of the different used solubilizing agents (isopropanol, glycerine and Seraqua™) are suitable for the active agent to reach the perilymph. The maximal concentration depends on the used solubilizing agent.

### Example 3: Further Pharmacokinetics Study of Different Active Agents

Pharmacokinetics study of different active agents has been conducted with one tailor-made formulation. The pharmacokinetic study consists of a single administration up to the eardrum on male Long Evans rats followed by assessment of the level of the active agent in the inner ear area (perilymph). Four formulations containing respectively 250 mg (5% w/w) of dexamethasone, 7,8-dihydroxyflavone (7,8-DHF), lidocaine and levodopa (L-DOPA) were assessed in this study. Apricot oil is present in the formulation as a solubilizing agent. α-(-)-bisabolol is used as diffusing agent.

The concentration of dexamethasone, 7,8-dihydroxyflavone (7,8-DHF), lidocaine or levodopa was quantified in the perilymph at 3 timepoints (1 hour, 4 hours and 8 hours).

### I. Materials and Methods

### 1. Formulations

The tested formulations are according to Table 6 below:

TABLE 6

| | Group | | | |
|---|---|---|---|---|
| Formulation | A | B | C | D |
| dexamethasone | 5% | — | — | — |
| 7,8-dihydroxyflavone | — | 5% | — | — |
| lidocaine | — | — | 5% | — |
| levodopa | — | — | — | 5% |
| α-(−)-bisabolol | 1% | 1% | 1% | 1% |
| benzyl alcohol | 1% | 1% | 1% | 1% |
| Aloe Vera | 5% | 5% | 5% | 5% |
| apricot oil | q.s. for | q.s. for | q.s. for | q.s. for |
| | 1 vial | 1 vial | 1 vial | 1 vial |
| | (88%) | (88%) | (88%) | (88%) |

The amounts are indicated in weight relative to the total weight of the formulation. The formulations are stored at room temperature.

2. Route of administration: 20 μl of each formulation are delivered in the ear canal of one animal in one administration.

3. Animals: 12 male rats Long Evans, were obtained from Janvier Labs, Le Genest St Isle, 53941 St Berthevin, France. Acclimation Animals arrived on site at least 5 days before the experiment to allow optimal acclimation. Selection: Animal health was examined on the day before starting the study, to ensure that only animals in good health were selected to enter the testing procedures. Identification: At the beginning of the study, animals were identified with a number on the tail. Each cage was labelled with a record card, indicating the test compound, the dose of administration and the identification of the animals. Housing: Rats were housed in cages (1354G (Tecniplast, Lyon), dim 595*380 mm (w*d)), in a room where the air was continuously filtered, thereby avoiding any contamination. During experiments, rats were caged in groups of five (300-400 g) at a constant temperature with a day/night cycle of 12/12 hours Animals received water (autoclaved water) and nutrition (ref A04C10), ad libitum.

4. Treatment and study design: The treatment was delivered into the ear canal and on the tympanic membrane on anesthetized rats (2.5% isoflurane). The exact volume of administration was determined during this first test and delivered (volume of 20 μL). Both ears were treated. After administration, the animal was kept on a heating pad for 5 minutes, positioned on its side to allow the compound to diffuse through the tympanic membrane. Then the second ear were administered, and the animal kept on its side for 5 minutes. Finally, the animal was kept on a ventral position until its recovery from anesthesia. The diffusion trough the tympanic membrane and into the inner ear and was assessed with the dosage of the cochlear fluid sampling. Tympanic bulla sampling technique: After sacrifice of the rats, the right tympanic bulla was collected from each animal and transferred to an Eppendorf tube, weighed individually and frozen at −20° C. until analysis. Perilymph sampling technique: After sacrifice of the rats and extraction of the tympanic bulla from the left ear, a volume of about 1 μL of cochlear fluid at the base of the cochlea was sampled ex vivo. Tubes were weighed individually before and after sampling, and immediately transferred to a −20° C. freezer.

5. Bioanalysis: The concentration of each compound in the perilymph and in the tympanic bulla was quantified using an exploratory LC/MS-MS assay method in outsourcing laboratory (Eurofins Vergeze, France). The concentration of amitriptyline in plasma was assessed.

### II. Results

Study results are reported in Table 7 below.

TABLE 7

| | | |
|---|---|---|
| | | Concentration |
| | Time of | (ng/mL) in |
| Formulation | sampling | perilymph |

| | | | |
|---|---|---|---|
| A (dexamethasone) | 1 | hour | 2217.5 |
| | 4 | hours | 208.84 |
| | 8 | hours | 74.003 |
| B (7,8-dihydroxyflavone) | 1 | hour | 355.99 |
| | 4 | hours | 1562.1 |
| | 8 | hours | 938.97 |
| C (lidocaine) | 1 | hour | 658.02 |
| | 4 | hours | 16321 |
| | 8 | hours | 30.192 |
| D (levodopa) | 1 | hour | 24.41 |
| | 4 | hours | 258.74 |
| | 8 | hours | 363.67 |

### III. Conclusion

Table 7 shows that the active agents (dexamethasone, 7,8-dihydroxyflavone, lidocaine and levodopa) are present within the inner ear (perilymph) after 8 hours from the administration onto the tympanic membrane.

This study on four formulations highlights the ability of the pharmaceutical composition of the invention to distribute the active agent into the inner ear after a local administration onto the tympanic membrane.

1-26. (canceled)

27. A method for the treatment of an inner ear disorder in a subject in need thereof;
wherein the method comprises a step of administration of a therapeutically effective dose of a pharmaceutical composition into the ear canal or onto the tympanic membrane of the subject; and
wherein said pharmaceutical composition comprises:
at least one active agent; and
a pharmaceutically acceptable vehicle comprising:
at least one solubilizing agent selected from the group consisting of vegetable oils, oil/water serum bases, glycerine, alcohols, and a mixture thereof, and
at least one diffusing agent selected from the group consisting of terpenes and derivatives thereof.

28. The method according to claim 27, wherein the at least one active agent is selected from the group consisting of repair agents, ionic channels antagonists, glutamate receptors antagonists, dopamine agonists or precursors, psychotropic drugs, antioxidant compounds, myorelaxant agents, vasodilators, diuretic compounds, other agents blocking signal transmission, nutritional supplements, anti-edema compounds, and a mixture thereof.

29. The method according to claim 27, wherein the at least one active agent is selected from the group consisting of repair agents, ionic channels antagonists, glutamate receptors antagonists, dopamine agonists or precursors, psychotropic drugs, antioxidant compounds, anti-edema compounds, and a mixture thereof.

30. The method according to claim 27, wherein the at least one active agent is selected from the group consisting of sodium channels antagonists, Tropomyosin Receptor Kinase (TRK) agonists, growth factors, and a mixture thereof.

31. The method according to claim 27, wherein the at least one active agent is selected from the group consisting of:
lidocaine;
psychotropic drugs selected from the group consisting of amitriptyline and cannabidiol;
glutamate receptor antagonists selected from the group consisting of dextromethorphan, gacyclidine, and selurampanel;
dopamine agonists or precursors selected from the group consisting of levodopa (L-DOPA), pramipexole, and ropinirole;
repair agents selected from the group consisting of 7,8-dihydroxyflavone (7,8-DHF) and neurotrophin-3 (NT3);
dexamethasone; and
a mixture thereof.

32. The method according to claim 27, wherein the at least one active agent is at a concentration ranging from 0.001% to 20% by weight based on the total weight of the composition.

33. The method according to claim 27, wherein said vegetable oils are selected from the group consisting of almond oil, apricot oil, arachis oil, camellia oil, castor oil, coconut oil, cottonseed oil, evening primrose oil, grapeseed oil, jojoba oil, maize oil, olive oil, rapeseed oil, rosehip oil, safflower oil, sesame oil, soybean oil, sunflower oil, medium chain triglycerides (MCT), wheat germ oil, and a mixture thereof.

34. The method according to claim 33, wherein said vegetable oil is apricot oil.

35. The method according to claim 27, wherein said alcohols are selected from the group consisting of propanediol, isopropanol, benzyl alcohol, ethanol, and a mixture thereof.

36. The method according to claim 27, wherein said terpenes and derivatives thereof are selected from the group consisting of sesquiterpene alcohols.

37. The method according to claim 36, wherein said sesquiterpene alcohols are selected from the group consisting of α-(-)-bisabolol, linalool, and a mixture thereof.

38. The method according to claim 27, wherein said pharmaceutical composition is in the form of ear drops or is comprised in ear drops.

39. The method according to claim 27, wherein the method comprises a step of administration of said pharmaceutical composition in the tympanic area of the subject.

40. The method according to claim 27, wherein said pharmaceutical composition is administered at a volume ranging from 5 μl to 100 μl per ear.

41. The method according to claim 27, wherein said inner ear disorder is selected from the group consisting of otosclerosis; non-obliterative and obliterative otosclerosis involving oval window; cochlear otosclerosis; acoustic neuroma; bilateral and unilateral sensorineural hearing loss; noise-induced hearing loss; aged-induced hearing loss; bilateral and unilateral conductive hearing loss; bilateral and unilateral mixed conductive and sensorineural hearing loss; sudden idiopathic hearing loss; drug-induced hearing loss; single sided deafness; ear infection; ototoxicity; drug-induced ototoxicity; herpes zoster oticus; labyrinthitis; purulent labyrinthitis; labyrinthine fistula; Labyrinthine dysfunction; vestibular neuronitis; excitotoxicity; autoimmune inner ear disease; acute unilateral vestibulopathy; vestibular neuronitis; benign paroxysmal positional vertigo; vertigo of central origin; Meniere's disease; Meniere's syndrome; and tinnitus.

42. The method according to claim 27, wherein said inner ear disorder is selected from the group consisting of hearing loss and tinnitus.

43. The method according to claim 27, wherein said at least one active agent is 7,8-dihydroxyflavone (7,8-DHF) or levodopa (L-DOPA); and wherein said inner ear disorder is tinnitus.

44. The method according to claim 27, wherein said at least one active agent is a growth factor or a Tropomyosin Receptor Kinase (TRK) agonist; and wherein said inner ear disorder is selected from the group consisting of hearing loss and tinnitus.

45. The method according to claim 44, wherein said Tropomyosin Receptor Kinase (TRK) agonist is a Tropomyosin receptor kinase B (TrkB) agonist or a Tropomyosin receptor kinase C (TrkC) agonist.

46. A device for the local administration of a pharmaceutical composition onto the tympanic membrane via the ear canal, said device comprising a pump, a reservoir, and a catheter channel;
wherein said reservoir comprises a pharmaceutical composition comprising:
at least one active agent; and
a pharmaceutically acceptable vehicle comprising:
at least one solubilizing agent selected from the group consisting of vegetable oils, oil/water serum bases, glycerine, alcohols, and a mixture thereof, and
at least one diffusing agent selected from the group consisting of terpenes and derivatives thereof.
